(19) United States
(12) Patent Application Publication
(10) Pub. No.: US 2024/0161892 A1
(43) Pub. Date: May 16, 2024

(54) Virtual Coach

(71) Applicant: BIOTRONIK SE & Co. KG, Berlin (DE)

(72) Inventors: Matthias WENZEL, Bremen (DE); Miro SELENT, Nuthetal (DE); Georg NOLLERT, Strasslach (DE); Dominic WIST, Berlin (DE)

(73) Assignee: BIOTRONIK SE & Co. KG, Berlin (DE)

(21) Appl. No.: 18/549,182

(22) PCT Filed: Feb. 22, 2022
(86) PCT No.: PCT/EP2022/054372

§ 371 Date: Sep. 6, 2023

(30) Foreign Application Priority Data
Mar. 10, 2021 (EP) 21161650.3

(51) Int. Cl. G16H 20/00; G16H 10/60; G16H 50/70

(52) U.S. Cl. CPC G16H 20/00; G16H 10/60; G16H 50/70

(57) ABSTRACT

The present invention relates to optimizing a health state of a patient. An apparatus comprises a) means for receiving a patient-specific value of at least one parameter; b) means for generating a profile for the patient based at least in part on the patient-specific value; c) means for selecting a stored profile out of one or more stored profiles of the patient and/or of other patients in a storage medium, the selecting based on a comparison of the stored profile and the generated profile for the patient, wherein the stored profile comprises a stored feedback and a stored patient-specific value of the at least one parameter; and d) means for providing feedback to patient based on the stored feedback.

### CROSS-REFERENCE TO RELATED APPLICATIONS

This application is the United States National Phase under 35 U.S.C. § 371 of PCT International Patent Application No. PCT/EP2022/054372, filed on Feb. 22, 2022, which claims the benefit of European Patent Application No. 21161650.3, filed on Mar. 10, 2021, the disclosures of which are hereby incorporated by reference herein in their entireties.

### TECHNICAL FIELD

The present disclosure relates to methods and apparatuses for optimizing a health state of a patient, such as implemented by a virtual coach.

### BACKGROUND

It is commonly known that in order to maintain and/or to recover a satisfying health state of a patient close caretaking and further steps may be required, particularly subsequent to a treatment of a patient in a hospital. Such caretaking may be based on physiotherapy, healthy food, sportive activity, rehabilitation, etc. Additionally, further check-ups may be required on a frequent basis.

Supervising and instructing such caretaking tend to be labor intensive (and raise costs) and further challenge the already strongly overloaded health system of many countries. Additionally, it may also be experienced as stressful for the patient if a large number of subsequent check-ups, visits to physicians and therapies are required to maintain or recover a satisfying health state.

Moreover, it is also often rather difficult for the patient and even for medical staff, in particular after a hospitalization, to exactly decide which actions (e.g., of everyday life) are harmful for the current health state and which kind of actions may yield a further and fast recovery of the health of the patient.

A compromise may be based on rather strongly standardized therapy patterns and standardized recommendations for the patients how to behave in order to enable recovering and/or maintenance of their health. However, such standardized proceeding neglects the individual needs and the individual recovery proceedings of the individual patients. Additionally, oftentimes it is difficult to create sufficient motivation of the patients to actively engage concerning such standard recommendations. Additionally, known proceedings are not really pre-emptive in a way that they motivate the patients to actively change their habits to avoid potential diseases, especially when they are at home and surrounded by everyday life.

Therefore, there is a need to improve the known ways to optimize the health management of a patient.

The present disclosure is directed toward overcoming one or more of the above-mentioned problems, though not necessarily limited to embodiments that do.

### SUMMARY

According to an aspect of the present disclosure, the above need is at least partly met by an apparatus for optimizing a health state of a patient. The apparatus may comprise means for receiving a patient-specific value of at least one parameter. The apparatus may further comprise means for generating a profile for the patient based at least in part on the patient-specific value, and it may comprise means for selecting a stored profile out of one or more stored profiles of the patient and/or of other patients in a storage medium, the selecting based on a comparison of the stored profile and the generated profile for the patient, wherein the stored profile comprises a stored feedback and a stored patient-specific value of the at least one parameter. Additionally, the apparatus may also comprise means for providing a feedback to the patient based on the stored feedback of the selected stored profile.

The at least one parameter may generally characterize the current state of the patient, e.g., it may include parameters characterizing the health state of the patient (e.g., blood pressure, heart rate) and/or indirect parameters associated therewith (e.g., humidity or temperature of the patient's environment), or it may relate to general patient data (e.g., date of birth, blood type). Stored profiles with e.g., positively confirmed feedback of patients for various states (e.g., a recommendation of one or more activities) may be accessed for that matter, which may include positively confirmed feedback (e.g., a recommendation of activities) that had turned out to be beneficial for the present state or at least for a similar state (e.g., as determined based on a comparison of the patient-specific value of the at least one parameter with the corresponding values stored in at least one stored profile). For example, stored profiles may reveal that for a previous patient with a similarly (high) body mass index and a similar age (and possibly also similar “soft” factors such as interests and hobbies), certain activities turned out to be effective for weight reduction (whereas for others, the patient's motivation may have been too low). These activities may then be selected for providing a feedback to the patient. Hence, a patient-specific feedback may be enabled, minimizing the required actions of medical staff and/or the patient and may enable an immediate reaction of the apparatus in response to any detected changes of the health state of the patient (which may be expressed by means of the received patient-specific value of the at least one parameter).

The patient-specific value of the at least one parameter may, for example, relate to one or more cardiovascular parameters (e.g., blood pressure, pulse rate) and/or cardio pulmonal parameters (e.g., a blood oxygen concentration), body temperature or any other patient-specific vital parameter. It may be measured by means of a medical device, e.g., based on data of an implanted device (e.g., a pacemaker, an implanted defibrillator, a cochlea implant, etc.).

Additionally or alternatively, the patient-specific value may also be based on entries in a database, a patient chart (e.g., including at least one of the above-mentioned parameters and/or further a detailed list of the administered medication, potential allergies and intolerances of the patient, an anamnesis, reports regarding earlier hospitalization, etc.). Further data sources may comprise a hospital information system (e.g., all information processing devices and systems in a hospital which may e.g., comprise doctors' letters, surgery reports, etc.), clinical data sets (e.g., information related to the health state of the patient, e.g., blood values, and further parameters which cannot directly be gained by means of sensors, e.g., the subjective wellbeing of the patient).

Additionally or alternatively, patient-specific data may also be retrieved over the internet (e.g., a GPS location of a patient and related environmental data (e.g., a temperature value, a humidity value, etc.)) and/or from a blockchain. The latter may provide the beneficial effect of providing a decentralized data storage medium which ensures data integrity (e.g., data consistency (data without contradiction)) and a decreased vulnerability (i.e., the chances for intrusion attacks and/or data losses and/or data piracy based on inadvertent and/or criminal activities may be minimized). Retrieving data over the internet may provide the advantage of correlating the at least one parameter (associated with the health of patient) to the current context of the patient, e.g., a temporary stay in a hot climate zone which may be determined based on GPS data (which may be determined by a smartphone or a smartwatch or any other suitable device) and the corresponding weather information which may be retrieved over the internet.

The patient-specific value of at least one parameter may also be retrieved by means of a mobile device which is not necessarily a medical device, e.g., a smartphone (app) or a wearable (e.g., a wearable such as a smartwatch or a step counter embedded in a (sports) shoe). The smartphone and/or wearable may comprise means for providing additional sensor data (such as a heart rate) or indirect patient-specific parameters (e.g., environmental parameters, such as a temperature and/or a humidity value).

The patient-specific value may be forwarded to the apparatus using an interface e.g., as described further below. The patient-specific value may relate to a static parameter (e.g., a birthdate of the patient and/or a blood type) and/or a dynamic parameter (e.g., at least one blood value, a heart rate, etc.).

For example, the apparatus may be implemented (e.g., embedded) in a server- or cloud-based system, e.g., comprising the storage medium (such as a database and/or a blockchain). The cloud-based system may be understood as a software-as-a-service (SaaS) and/or an infrastructure-as-a-service (IaaS) implementation. The server system may provide hardware and/or software resources to perform the functions of the present disclosure. Such resources may comprise at least one transient and/or non-transient storage medium and/or at least one processor and/or at least one medium for communication (e.g., one or more interface). The server system may be implemented as part of a hospital information system and/or a remote server system (e.g., in a datacenter which may be accessible over the internet).

The means for receiving the patient-specific value may be based on an interface. An interface may generally refer to a hardware and/or a software interface. A hardware interface may be understood as e.g., a USB connection, an ethernet connection or any other suitable hardware interface which allows a reception of a patient-specific value of at least one parameter. The hardware interface may be based on a wireless (e.g., Wi-Fi, 5G, LTE, Bluetooth or any other suitable standard and/or air interface) and/or a wired connection (e.g., a copper wired connection), e.g., via the internet. Additionally or alternatively, the interface may also be embedded in software and may relate to various protocols (e.g., IP, TCP, UDP, I2C, GPIO, etc.). The software interface may further be based on the communication of web sockets.

The apparatus may generally communicate (telemetrically and/or continuously and/or bidirectionally) with a smartphone, a wearable, e.g., a smart watch, a fitness tracker, a sports device (e.g., a treadmill, an exercise bike, a rowing machine), a smart home device (e.g., a smart speaker), etc. of the patient, by means of which the feedback is ultimately provided to the patient. This communication may occur via an interface of the apparatus as described above.

Further, it is also conceivable that the apparatus is implemented e.g., in or as a mobile device, such as a smartphone, or another wearable adapted to be carried by the patient (e.g., a smart watch, a fitness tracker), or a sports device (e.g., a treadmill, an exercise bike, a rowing machine), a smart home device (e.g., a smart speaker), etc. The apparatus may then be adapted to communicate with a server-based system which may comprise the storage medium in which the stored profiles are stored, e.g., using an interface as described above. The mobile device may then itself provide the patient-specific value. Additionally or alternatively, the mobile device may comprise an interface as described above, to receive values from other sources. In case of a local implementation of the apparatus (e.g., in a smartphone of the patient), the apparatus may use the interfaces typically present in the local device (e.g., touchscreen, speaker) to provide feedback to the patient. Additionally or alternatively, the apparatus may communicate with a smartphone, a wearable etc. as described above to provide the feedback to the patient.

It is also conceivable that a medical device is comprised by the apparatus, e.g., the apparatus may be implemented by one or more processors in the medical device. The apparatus/medical device may then be adapted to communicate with a server-based system which may comprise the storage medium in which the stored profiles are stored. The apparatus may e.g., be part of an implant (e.g., as a system on a chip (SoC) or any other integrated circuit (IC)). Alternatively, it may be embedded in the implant by means of an implementation as a respective software (method), e.g., on an EPROM, EEPROM or FPGA.

The patient-specific value of a least one parameter may be based on first, second and/or third rank data. First rank data sets may refer to the above-mentioned parameters which are directly related to the patient (e.g., a value of the blood pressure). Second rank data may relate to parameters which are based on first rank data but which are further processed, e.g., to determine heart-specific parameters in dependence on the blood pressure or the calculation of a risk for heart attacks in dependence on markers which can be determined from the investigation of blood samples (e.g., troponin). Third rank data may be based on second rank data but further processed, i.e., on a higher level of abstraction compared to second and first rank data.

Optimizing in the context of the present application may be understood as (iteratively) providing feedback to the patient such that a wellbeing of the patient is improved. The optimization may be patient-specific, i.e., different patients may require different feedbacks. An optimized health state of a patient may be based on at least one value of an objective parameter (e.g., a target blood pressure of 120/80 mmHg and/or a resting heart rate of 60 beats per minute) or may be based on at least one value of a subjective parameter, e.g., an individual feeling of a patient in response to providing a feedback to the patient (this may additionally involve a confirmation by the patient that the provided feedback has been implemented), which may refer to a level one or more of pain, vertigo, nausea, etc., and/or feedback by medical staff.

Optimizing in the context of the present invention may not only refer to quantitative patient-specific values but may also relate to qualitative values (e.g., an indication of the patient that the patient suffers of vertigo or sickness). In such a case, the comparison of profiles may comprise finding a profile among the stored profiles which relates to a similar subjective feeling of the patient and/or another patient. The associated feedback, which had recently led to an improvement of the wellbeing of the patient may be selected for the present case and provided to the patient.

Such an apparatus for optimizing the health state of the patient may even allow a patient to leave hospital early as it may allow for further caretaking at home (e.g., by means of providing a certain feedback if e.g., the blood pressure approaches a regime which is considered as harmful for the patient), in an environment the patient is familiar with. This may also increase the general wellbeing and satisfaction of the patient.

What is more, and as will be outlined in further detail below, an updated profile may be generated for the patient based on the feedback, and—depending on the actual evolution of the patient's state—this profile may then be added as a further stored profile to the storage medium, such that it will be available for further feedbacks for the patient and/or other patients. Hence, a self-learning apparatus may be provided. Such a procedure may be performed in closed loop operation with the goal of maximizing the wellbeing of the patient while minimizing the actively required efforts of the medical staff and/or the patient. A close loop operation in the given context may be understood as iteratively performing the steps as described above with the goal of converging the current health state of the patient towards a value set which may be perceived as satisfactory. The value set may e.g., comprise a blood pressure interval which may be seen as at least healthier than a current blood pressure measurement (which may be indicated by the received patient-specific value).

Generating a patient-specific profile may (in an exemplary manner) be understood as storing the received patient-specific value(s) of at least one parameter (e.g., to a virtual flashcard). The profile may further be assigned with a patient-specific ID (which may be anonymous). Equipping the generated profile (the virtual flashcard) with a patient-specific ID, may further provide the beneficial effect of a transparent assignment of the generated profile to a particular patient. Furthermore, the generated profile may then not only comprise a single temporary snapshot of the state of the patient (which may be represented by the received patient-specific value of at least one parameter) but it may comprise several snapshots of the state of the patient (which may be represented by repeatedly received patient-specific values of at least one parameter). The latter may then allow to review the temporal evolution of the state of the patient, possibly with a high frequency and/or quasi-continuously (with updates e.g., every day, every hour, every minute, every second, etc.). Alternatively, it may also be possible that a new patient-specific profile is generated for each of the one or more snapshots of the health state of the patient. In such an application, the generated patient-specific profiles may ex post be assignable to a particular patient by means of the patient-specific ID. Monitoring the time evolution of the patient-specific value may provide the possibility of providing a feedback to the patient only if a certain deviation of the patient-specific value from an interval (which is considered as healthy) is recognized. This may in particular allow to avoid any deteriorating effects on the health of the patient on long time scales (e.g., several days, several weeks, several months, several years, etc.) and/or if it is occurring repeatedly (e.g., every week, every month, every year, etc.) over a longer time scale but which may not ultimately be harmful if occurring as a single event (e.g., an increased value of LDL cholesterol in a blood sample). In the case of a constantly increased value of LDL cholesterol in a blood sample, the apparatus may provide the patient with the feedback that e.g., cholesterol containing food should be avoided and/or that the patient should change the respective eating habits which led to the increased value of LDL cholesterol.

The means for selecting may further be adapted to select a stored profile based on a positivity value associated with the stored feedback of the stored profile. The positivity value may be stored in the stored profile. The positivity value may be associated with a likelihood that the feedback to be provided to the patient, which is stored in the profile (a profile may comprise one or more feedbacks each having a positivity value), may lead to an increase of a health state of the patient. The likelihood may be expressed as a probability value wherein the probability value may be any value in between 0 and 100%. A value of 100% may indicate that the respective feedback associated with the stored profile may lead to an increase of the health state of the patient with a probability of 100%. If a feedback has led to an improvement of a health state of a patient but did not result in such improvement with another patient, the positivity value may be 50%. Alternatively, the positivity value may also be scaled to an interval of −1 and 1 (or −100% to +100%), with −1 indicating a deterioration of the health state of the patient and 1 indicating an improvement of the health state of the patient. The positivity value may also be based on a binary value set (e.g., − and +) or any other suitable value set.

The comparing of the generated profile for the patient with stored profiles may lead to feedbacks that are found in more than one stored profile. If, in addition, several or all of these multiple stored feedbacks are also equipped with a positive positivity value (indicating an expected improvement of the health state of the patient), the feedbacks (associated with the stored profiles) may be interpreted as more reliable. In other words, if multiple stored profiles, which comprise similar patient-specific values, are assigned with a positivity value which indicates that the related feedbacks to be provided to the patient have (historically) led to an increase of the health state of the patient, the respective feedbacks may be seen as more reliable due to the increased statistical support. Therefore, a stored profile may not only be selected based on requiring a certain positivity value but also based on a certain statistical support (i.e., the (at least one) feedback must have led to a positive result for various profiles). The statistical support may comprise requiring that at least two, three, four, five, and/or ten different stored profiles need to be assigned with a certain positivity value (e.g., at least 50%). Alternatively, it may also be possible that the selection requires that a certain feedback has led to an improvement of the health of the patient with a certain frequency, e.g., in more than 80%, more than 90%, more than 99% or more than 99.9% of all stored profiles comprising this feedback.

The stored profiles may be historic profiles, i.e., profiles which were accumulated in the past after the provision of feedbacks and evaluation of the effect on the health state of the respective patient (e.g., by means of determining an associated positivity value). The evaluation may be based on retrieved patient-specific values (e.g., a decrease of the blood pressure may be interpreted as a positive change of the health state of the patient after a hypertonia derailment) and/or input by the patient as it will also be described in further detail below. The stored profiles may relate to the same and/or at least one other patient (e.g., the profiles may be evidence-based, associated with a positivity value concerning the effect of their feedback, e.g., whether the feedback of the profile was beneficial or detrimental, possibly with a value indicating a likelihood).

The comparison of the profile for the patient with stored profiles of the patient and/or of other patients in a storage medium may be directed to comparing a single patient-specific value of at least one parameter to a stored patient-specific value of the at least one parameter. Alternatively, it may also possible to compare the patient-specific values of more than one parameter to stored corresponding values of these parameters. Additionally or alternatively, the patient-specific value of a parameter may also be represented as a vector. It may be possible that each row of the vector may represent a temporal snapshot of the patient-specific value of the at least one parameter which may thus facilitate the tracking of temporal evolutions of the at least one parameter.

The stored profiles may have been generated based on patient-specific data sets which are related to the same patient and/or may be based on patient-specific data sets of at least one other patient. Additionally, the stored profiles may also be used to interpolate data sets which were not retrieved continuously, e.g., due to communication errors or organizational issues (e.g., a blood value which is not determined periodically due to holidays or increased workload). The comparison may also be understood as a self-learning system, in particular, an artificial intelligence (AI) as it may be possible to provide the patient with feedback based on the current patient-specific value of the at least one parameter (associated with the state of the patient) without any further input, e.g., from a medical staff and/or the patient.

It is also conceivable that, instead of feedback contained in one of the stored profiles, the apparatus is adapted to generate new feedbacks based on one or more stored feedbacks. For example, if two feedbacks are stored with high positivity values in profiles with patient-specific parameter values similar to those of the generated profile, e.g., a “mean value” of the two feedbacks may be provided to the patient, that may be selected by AI algorithms. For example, if feedback may be interpolated. For example, for patients with a higher BMI, a less straining exercise program may initially be suggested compared to patients with lower BMI, as indicated by the stored profiles (in order not to overly strain joints, for example). Then, the apparatus may select an exercise program with an “intermediate” strain level if the generated profile indicates an “intermediate” BMI.

The comparison may also comprise the determination of similarities between the profiles and, in particular, the determination of a rank of similarity. A rank “1” similarity may relate to a similarity or even identity of all relevant patient-specific parameter values in the profile which are involved for the comparison. Which values are considered as relevant values may be defined by the specific application, e.g., based on the current circumstances of the patient (e.g., a post-operative patient with one or more health constraints, e.g., the requirement that certain foods should be avoided). As an example, if the present invention is used to suggest eating habits (and to optimize nutrition habits), parameters (which may be comprised by the generated profile for the patient) which may indicate a broken bone may not be regarded as relevant. However, if a previous heart attack has been documented in the anamnesis, such information may be considered as highly relevant. Rank “two”, “three”, . . . to rank “n” similarities may refer to a similarity in all but one, two, . . . n−1 parameter values. This distinction of the profiles may allow the determination whether a patient with a similar health state has already been documented (and stored). For example, profiles with “rank 1”, “rank 2” etc. compared to the generated profile for the patient may be displayed to the medical staff, together with statistical data concerning a frequency of certain deviations in the parameter values and/or the corresponding at least one feedback.

Providing a feedback to the patient may be understood as e.g., providing a suggestion to the patient how to change individual habits to increase the health state. Such suggested changes in individual habits may e.g., by a suggestion to avoid certain foods, increase sports activities, stop smoking, etc. The suggestions may further be based on detailed plans how to increase the health state. Such plans may be developed by the patient and/or the medical staff. The suggestions may comprise detailed exercises, recipes for cooking, suggestions when to go to sleep, suggestions for a certain medication, etc. The feedback may not only comprise an active action but may also include a suggestion to avoid certain actions (e.g., avoid certain foods as there may be a known intolerance or alcoholic beverages if e.g., pre-existing liver diseases are known). It may be possible that only a single feedback is provided to the patient. However, it may also be possible that a plurality of feedbacks is provided to a patient, e.g., at the same time or one after the other. A plurality of feedbacks may e.g., comprise a training plan in order to lose weight.

It may be possible to provide a single feedback to the patient (e.g., suggesting that the patient should go running once a week for 60 min) but may also comprise more than one feedback (e.g., suggesting the patient to go running once a week for 60 min and in addition suggesting the patient to avoid certain foods).

The feedback may be provided by means of a smartphone app, by means of a wearable (e.g., a smart watch), a sports device (e.g., a treadmill, an exercise bike, a rowing machine, etc.), by means of an oral suggestion (e.g., by means of a smart home device such as a smart speaker), etc. with which the apparatus may be in communication (or in which the apparatus may be implemented).

In some examples, the means for providing the feedback is adapted such that, if the stored positivity value is negative, the feedback provided to the patient suggests to the patient to avoid an activity associated with the stored feedback. For example, if a certain diet has turned out to be negative for previous patients (and/or the same patient) with a similar profile, the present patient can be informed to avoid such diet.

In some examples, the means for providing the feedback is adapted such that, if the stored positivity value is positive, the feedback provided to the patient suggests an activity associated with the stored feedback.

In some examples, the apparatus may comprise means for obtaining a positivity value associated with the feedback provided to the patient. Additionally, the apparatus may comprise means for updating the profile for the patient based at least in part on the obtained positivity value. Further, the apparatus may comprise means for storing the updated profile for the patient in the storage medium. Hence, new profiles may be generated and made available for further use of the apparatus and/or other apparatuses of other patients having access to the storage medium.

The means for obtaining the positivity value may further comprise means for receiving an update of the patient-specific value of the at least one parameter and/or of a patient-specific value of at least one further parameter (or an update of the further parameter). The feedback may be provided to the patient (and possibly applied by the patient which the patient may optionally indicate to the apparatus). The apparatus may then receive an update of the patient-specific value and an evaluation may be made whether the patient-specific value has led to an increase or a decrease of the health state of the patient. If an increase of the health state of the patient is determined, the positivity value may be chosen to represent the increase of the health state of the patient. If a decrease of the health state is determined, the positivity value may be chosen such that a decrease of the health state of the patient is represented due the provided feedback. Potential value sets for the positivity value have been described above. The positivity value may be obtained based on an evaluation of the same patient-specific value of at least one parameter (in response to which the feedback had been provided to the patient beforehand) and/or may be based on a patient-specific value of at least one other parameter. As an example, if a feedback has been provided to the patient, e.g., based at least in part on an increased blood pressure, the obtaining of the positivity value may also be based on an evaluation of a pulse rate.

In addition to storing the updated profile, the positivity value determined for the updated profile may also be used to adjust the positivity value of the stored profile whose stored feedback was selected. For example, if the patient's health state improved (deteriorated) after applying the feedback, the positivity value of the feedback may also be reduced in the stored profile whose feedback was selected. Still, additionally or alternatively, it is conceivable to generate a cumulative stored profile, e.g., a profile which includes the selected feedback and which includes two (or more) (sets of) patient-specific values: that of the generated updated profile and that of the already stored profile. Then, an average positivity for the selected feedback, and the two (or more) (sets of) patient-specific values may be stored in the cumulative profile.

The means for obtaining a positivity value may further comprise means for obtaining an input from the patient (e.g., direct feedback). The input may be based on an input of the patient that the feedback has been applied and/or that the application of the provided feedback has led to an increase of the health state of the patient. As a result, a respective positivity value may be obtained which indicates that the (application of the) respective feedback has led to an improvement of the health state of the patient. The direct feedback of the patient may be received by means of a user interface, e.g., a smartphone app (in which a GUI may be foreseen to receive an input by the patient by means of pressing an implemented button which indicates an exemplary increase of the health state of the patient), a device (e.g., a wearable, such as a smartwatch) which may be in wireless and/or wired connection with the apparatus, a voice recognition system (e.g., a natural language processing system) or any other suitable implementation for obtaining feedback. The feedback may also be obtained indirectly by means of an input of the medical staff. For example, the apparatus may be given a corresponding input that the feedback has successfully been applied (e.g., directly by the medical staff or via another device accessible by the medical staff such as a smartphone which is also understood to include tablets). This may be useful in linking a perceived change in patient-specific values to a certain feedback provided to the patient.

The apparatus may further comprise means for providing a feedback to the patient based on the positivity value. Based on the selected stored profile, which may contain one or more feedbacks each having a positivity value, it may be decided whether to provide one of the feedbacks (associated with the selected stored profile) to the patient or not. The decision may be based on a threshold (which may be pre-defined) for the positivity value. It may be required that the positivity value needs to be above a certain threshold (or below a certain threshold). In such a case, the feedback associated with the positivity value (positive or negative feedback) may be regarded as promising to, when applied by the patient, yield to an increase of the health state of the patient. If the threshold cannot be exceeded, the associated feedback may be rejected. A positive feedback may e.g., include a suggestion to take a particular action in order to increase the health state of the patient. Such a suggestion may e.g., be to increase sports activities, eat more vegetables, etc. If the positivity value is negative, the negative feedback may relate to a suggestion which pursues an avoidance of certain actions, such as e.g., avoiding certain foods, avoid smoking, etc., as outlined above. The means for receiving the one or more patient-specific value may comprise means for repeatedly receiving updates of the at least one patient-specific value. This may in particular provide the advantage that the health state of the patient may continuously be tracked. Any deviation from a target range may be subject to a quick detection and the respective feedback to the patient may be provided which may be helpful to return to the target range. Such a target range may e.g., comprise a target blood pressure of 120/80 mmHg and may be patient-specific (e.g., a higher target blood pressure may be defined for patients suffering hypertonia) and or generically applicable for a wide range of patients. Repeatedly receiving updates in that context may refer to receiving updates periodically. Periodically receiving updates may relate to receiving updates on a regular time scale, e.g., every second, every minute, every hour, every day, once per year or even longer time scales. Additionally or alternatively, the updates may also be received quasi-continuously, e.g., approximately every day, every hour, every minute or even on shorter timescales etc. In particular, the updates may also be received discontinuously and/or on an irregular time basis, e.g., as a result of an explicit request by e.g., the medical staff and/or the patient. Such a request may be based on a sudden change of the state of the patient. For example, the blood pressure of the patient may be tracked on long time scales (e.g., once per day or once per hour, for the sake of monitoring the state of the patient) and may switch to immediately measuring the blood pressure of the patient and/or measuring repeatedly in shorter intervals (e.g., every second or every minute), if it is detected that the state of the patient has deteriorated (i.e., the blood pressure may have harmfully increased). After a normalization of the blood pressure is detected, the frequency of receiving updates may switch back to longer time scales (e.g., to avoid an undue amount of data production).

The means for selecting a stored profile may comprise means for calculating a metric for the stored profile, wherein the metric is calculated between the value of the at least one parameter in the generated profile for the patient and the stored patient-specific value of the at least one parameter in the stored profile.

The calculation of the metric may be performed based on one patient-specific value of one parameter in the generated profile for the patient and one stored patient-specific value of the respective one parameter in the stored profile. Alternatively, the calculation of the metric may be based on a plurality of patient-specific values for a plurality of parameters. For example, patient-specific values for each of several parameters in the generated profile for the patient and corresponding stored patient-specific values may be used. Additionally or alternatively, also patient-specific values taken at different instances in time may be used for the metric. In case profiles do not relate to a single instant in time but include temporal evolution of a patient's state (as outlined above), the metric may relate to a deviation of stored patient-specific values from those in the generated profile for the patient during the course of time. Hence, stored profiles may be identified, based on the metric, which describe a similar trajectory of the state of a patient over time. The calculation of the metric may be based on some or all of the patient-specific values of some or all available parameters of the generated profile and/or the stored profile, respectively. Optionally, it may also be possible include weighting factors into the calculation of the metric. Such a weighting of one or more parameters may be beneficial to weight certain vital parameters (represented by the weighting factors) which may in particular be harmful to the health state of the patient when exceeding certain intervals (e.g., the blood pressure for patients sensitive to strokes).

The means for calculating may be adapted to calculate a metric for a plurality of the stored profiles, and the means for selecting may be adapted to select a stored profile for which the metric is minimized. Hence, the best “match” may be identified. The calculation of the metric may be performed for selected stored profiles or may be calculated for all available profiles. For example, the calculation of the metric may be limited to stored profiles fulfilling certain predetermined conditions. For example, if two or more parameters are available in the generated profile and the stored profiles, the metric may be calculated only for stored profiles with one of the parameters fulfilling a certain predetermined criterion, e.g., the metric may be limited to profiles of patients with similar or same age, of patients having similar diseases (having experienced at least one stroke, diabetes), etc.

The means for selecting the stored profile may be adapted to select the stored profile, (only) if the metric is below a threshold. The threshold may be a pre-defined value which may be defined by the medical staff and/or the patient and may be understood as a value which defines how close the at least one patient-specific value for at least one parameter in a stored profile needs to be to the corresponding at least one value for at least one parameter in the corresponding generated profile to interpret a stored profile as a “match”. The threshold may be a global value (i.e., identical for all patients) or may be a patient-specific value, which allows an adaption of the selection process according to the individual needs of the patient.

This threshold criterion may be applied additionally or alternatively to the selection of the “minimum”, as outlined above. For example, a selected minimum may need to be below the threshold in order to be selected. Alternatively, a profile may immediately be selected if its metric is below threshold, regardless of whether there may be a profile with a lower metric value. Optionally, also the positivity value of the feedbacks of the profile may then be looked at: If no positivity value of the selected profile is above a positivity threshold (or below a negative threshold), the profile may be discarded. The further analysis may then select a further profile whose metric is below threshold. It may then be verified whether this profile comprises feedbacks with better positivity values, e.g., above or below threshold. This way, stored profiles may be iteratively checked.

It may also be possible, that a feedback may only be selected, if the patient-specific value of the at least one parameter deviates from a threshold. For example, a feedback may only be selected, if e.g., a pulse rate deviates by more than e.g., 10 bpm from an interval which is defined as “normal” (e.g., a pulse rate between 50-90 bpm at rest). Alternatively and/or additionally, a feedback may be selected based on an input of the medical staff and/or the patient. This may e.g., be beneficial if the subjective wellbeing of the patient may change (e.g., due to a suddenly occurring vertigo). The dedicated threshold for the maximum allowable deviation may be patient-specific (by e.g., considering pre-existing diseases) or may be a generic value.

It is therefore a further aspect of the disclosure to provide an apparatus which comprises means for receiving an update of a patient-specific value of a least one parameter (or repeatedly receiving updates). A profile for the patient may be updated based on the updates of the patient-specific value. The apparatus may comprise means for providing feedback as described herein, which provides a feedback (only) if the patient-specific value exceeds one or more predetermined values. For example, the values may be predetermined as certain deviations (+/−) from an initial value of the patient-specific value.

The storage medium may be a blockchain. The apparatus may be adapted to store the generated profile for the patient in a blockchain. Storing the generated profile in a blockchain may in particular provide the advantage of increasing the data integrity and redundancy (as outlined above) associated with the generated profile.

In an exemplary embodiment, the apparatus for optimizing a health state of a patient comprises a) means for receiving a patient-specific value of at least one parameter, b) means for generating a profile for the patient based at least in part on the patient-specific value, c) means for selecting a stored profile out of one or more stored profiles of the patient and/or of other patients in a storage medium, the selecting based on a comparison of the stored profile and the generated profile for the patient, wherein the stored profile comprises a stored feedback and a stored patient specific value of the at least one parameter, and d) means for providing feedback to patient based on the stored feedback. Furthermore, the means for selecting a stored profile comprises means for calculating a metric for the stored profile, wherein the metric is calculated between the value of the at least one parameter in the generated profile for the patient and the stored patient-specific value of the at least one parameter in the stored profile, wherein the means for calculating is adapted to calculate a metric for a plurality of the stored profiles, and the means for selecting is adapted to select a stored profile for which the metric is minimized, and wherein the means for selecting the stored profile is adapted to select the stored profile, if the metric is below a threshold. Furthermore, if no suitable stored profile can be determined whose calculated metric is below a pre-defined threshold, an augmented patient-specific profile is generated comprising the generated patient-specific profile and at least one additional parameter associated with the state of the patient.

This embodiment solves the problem of improving automated feedback, especially when there is a lack of information/matches. The features of this embodiment make it possible to obtain information/feedback in an automated manner, specifically by means of a guided human-machine interaction.

The present disclosure further comprises a method for optimizing a health state of a patient. The method may comprise receiving a patient-specific value of at least one parameter and it may comprise generating a profile for the patient based at least in part on the patient-specific value. The method may further comprise selecting a stored profile out of one or more stored profiles of the patient and/or of other patients in a storage medium, the selecting based on a comparison of the stored profile and the generated profile for the patient, wherein the stored profile comprises a stored feedback and a stored patient-specific value of the at least one parameter, and it may comprise providing feedback to the patient based on the stored feedback.

The method may further include selecting the stored profile based on a stored positivity value associated with the stored feedback of the stored profile. This method step is performed in analogy to the above-mentioned apparatus.

The method may further comprise any of the further steps described herein, even if they are specifically described with respect to an apparatus. The method steps may be carried out iteratively and/or in a closed loop operation.

The present disclosure further comprises a computer program which may comprise instructions which may cause a computer to implement the method steps described herein, and/or the means as described herein, when the instructions are executed.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media includes both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

It is further noted that the present invention is not limited to the specific feature combinations expressly listed herein, which are only understood as examples. Other features and/or feature combinations may also be possible.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The following FIGURE is provided to support the understanding of the present invention:

FIG. 1 Illustration of a flow chart of a possible implementation of the virtual coach apparatus/method according to the present disclosure.

### DETAILED DESCRIPTION

The following detailed description of the present invention and further embodiments described therein are provided to facilitate the understanding of the present invention.

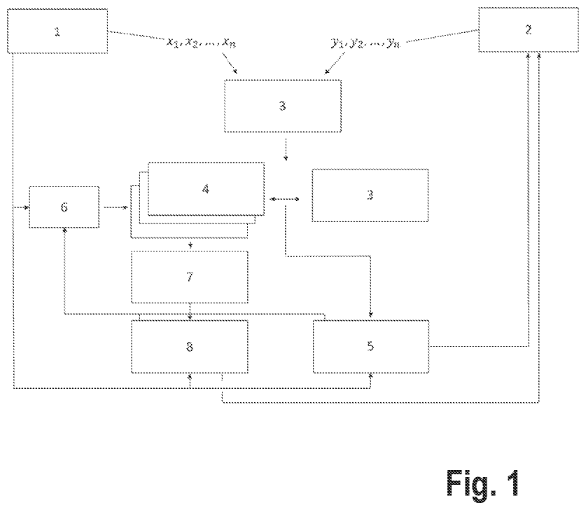

FIG. 1 shows a flow diagram of an exemplary embodiment of the steps carried out by an apparatus or a method according to the present disclosure. A state of a patient 1 is described by at least one patient-specific value of at least one parameter x₁, . . . , xₙ. The at least one parameter may represent at least one of a cardiovascular parameter (e.g., blood pressure, pulse rate) and/or a cardio pulmonal parameter (e.g., a blood oxygen concentration), body temperature or any other patient-specific vital parameter. Alternatively, the at least one patient-specific value may at least be based on entries in a database, a patient chart (e.g., at least one of the above-mentioned parameters and/or further a detailed list of the administered medication, potential allergies and intolerances of the patient, an anamnesis, reports regarding earlier hospitalization, etc.), data of an implanted device (e.g., a pacemaker, an implanted defibrillator, a cochlea implant, etc.). Further data sources may comprise a hospital information system (e.g., all information processing devices and systems in a hospital which may e.g., comprise doctors' letters, surgery reports, etc.), clinical data sets (e.g., information related to the health state of the patient, e.g., blood values, and further parameters which cannot directly be gained by means of sensors, e.g., the subjective wellbeing of the patient). Additionally or alternatively, patient-specific data may also be retrieved over the internet, e.g., a GPS location of a patient and related environmental data (e.g., a temperature value, a humidity value, etc.) and/or from a blockchain.

A smartphone 2, as described herein, may be used to provide at least one feedback y₁, . . . , yₙ to the patient. The smartphone may run an app (e.g., a virtual coach app) which provides the functionality as described herein. The at least one feedback may e.g., be related to a certain suggestion for a changing a habit, a suggestion for a medication dose, the suggestion to avoid particular foods, etc.

A patient-specific profile 3 may be generated based on the at least one patient-specific value of the at least one parameter, and possibly with previously provided feedback to the patient 1 (if any). The patient-specific profile may be understood similarly as a flashcard which comprises at least one parameter that may characterize (a state of) the patient (encoded as the at least one patient-specific value). The generated patient-specific profile 3 may additionally be assigned with a patient-specific ID. A patient-specific ID may be understood as a distinct (but anonymous) indicator used to unambiguously identify a patient. The patient-specific profile 3 may either be generated once per patient and may comprise a temporal evolution of the at least one patient-specific value which e.g., facilitates the evaluation whether a feedback has led to a desired change of the at least one patient-specific value. Alternatively, it may also be possible that the patient-specific profile 3 only comprises a snapshot of the current at least one patient-specific value. By additionally assigning a patient-specific ID to the generated patient-specific profile 3, it may ex post be possible to assign the at least one patient-specific profile 3 to the respective patient.

The generated patient-specific profile 3 may further be compared to at least one stored profile 4. The at least one stored profile 4 may be based on evidence-based (historic) data sets, may relate to the same patient or at least one other patient as described above. The comparing may e.g., comprise a comparison of the at least one patient-specific value of at least one parameter of the generated patient-specific profile to a stored patient-specific value of at least one parameter in the at least one stored profile. The comparison may also comprise a calculation of at least one metric between the at least one patient-specific value of the generate patient-specific profile and the stored patient-specific value in the at least one stored profile, which may be understood as the calculation of a difference between the respective values. A stored profile and the generated patient-specific profile may be understood as “similar” or “matching” if the calculated difference is below a threshold. The threshold may be a pre-defined value, identical for all patients, or may be a subjective value which is particularly chosen for a certain patient. The latter may in particular be used to consider pre-existing diseases which may e.g., cause a ubiquitous offset of the blood pressure of the patient with respect to a blood pressure which is generally regarded as normal and healthy. If two profiles are considered as similar, the similar stored profile may be selected.

Each stored profile may also comprise one or more feedbacks, each associated with a positivity value which represents a likelihood that the associated at least one feedback may yield an improvement of the health state of the patient corresponding to the patient-specific values stored in the profile. A selected stored profile may only be further processed if the positivity value is above (below) a (negative) threshold wherein the threshold may be defined by the medical staff and/or the patient.

The selected feedback may be suggested to the patient 1 by means of the smartphone 2.

Before suggestion to the patient, it may also be implemented to present updated profile 5 to a member of the medical staff, e.g., a smartphone of the medical staff. The medical staff may be asked to confirm the selected feedback associated with the updated profile 5 prior to suggesting it to the patient 1. The medical staff may also reject the updated profile 5 if it is, e.g., expected that the feedback would not lead to an improvement of the health state of the patient and/or may be considered as dangerous.

The feedback associated with the selected stored profile may be used to generate an updated profile 5, including this feedback and the patient-specific value of the at least one parameter of the generated profile 3 for the patient. After providing the feedback to the patient by means of the smartphone 2 (and optionally indicating its realization to the apparatus by the patient), the at least one patient-specific value may be retrieved from the patient 1, e.g., by means of a blood pressure measurement. The at least one value may be retrieved repeatedly (e.g., periodically or quasi-periodically as described above) or may only be retrieved once. It may automatically be detected whether the provided and/or applied feedback has led to an improvement of the health state of the patient, e.g., if the measured blood pressure has returned into an interval which is considered as not harmful/healthy for the patient. Additionally or alternatively, this assessment may be made by means of an external confirmation whether the at least one applied feedback has led to an improvement of the health state of the patient. This may comprise the subjective feeling of the patient input via smartphone 2. Hence, a positivity value may be determined.

An updated profile 6 (comprising the least one patient-specific value, possibly update(s) thereof, at least one (ideally confirmed) feedback and the positivity value) may be then be generated and stored together with the at least one stored profile 4. Optionally, the updated profile 6 may only be stored if it is confirmed that the feedback has been applied (i.e., if it is determined that the patient has changed a certain habit and/or if the patient itself confirms the application of the feedback, e.g., by means of a smartphone app). The updated profile 6 may also be stored with a positive/negative positivity value if the medical staff confirms/rejects the updated profile 5.

If it is detected that the applied feedback deteriorates the state of the patient, the smartphone 2 may either be advised to provide the patient with a preceding feedback, it may provide a feedback to revoke the previous feedback, and/or may be advised to provide the patient with a default feedback which may e.g., be defined by the medical staff and/or the patient. It may also be possible that the updated profile 6 is stored, even if it deteriorates the health state of the patient. In such a case, the positivity value may be assigned accordingly (i.e., may be assigned with a value that indicates the deterioration). Hence, the updated profile 6 (which deteriorates the health state of the patient), may be recognized as potentially harmful for the patient if it is selected in any of the future iterations of the described sequence. Specifically, a selected feedback may be cross-checked for profiles indicating this feedback as harmful. If this cross-check results in a hit, it may also be provided to the medical staff/patient, and/or the feedback may not be provided to the patient by means of the smartphone 2. Also, if an updated profile 6 with feedback comprising a negative positivity is stored, all other patients (or their smartphones) may be alerted immediately which have been provided with the same or similar feedback (e.g., based on the same or similar patient-specific values).

If the comparison of the generated patient-specific profile 3 with the at least one stored profile 4 does not result in a “match” (e.g., if no suitable stored profile can be determined whose calculated metric is below a pre-defined threshold), an augmented patient-specific profile 7 may be generated. The augmented patient-specific profile 7 may comprise the generated patient-specific profile 3 but may further comprise at least one additional parameter associated with the state of the patient. By means of including the at least one additional parameter, it may be possible to explain the at least one difference (i.e., the reason why no stored profile can be found which represents a “match” to the generated profile 3) between the generated profile 3 and the at least one stored profile.

After generating the augmented profile 7, a new comparison between the augmented profile and the at least one stored profile may occur. The comparison may comprise the calculation of at least one metric. If a match can be found, a stored feedback of the stored profile that represents the “match” is selected as the feedback, and an updated profile 8 may be generated. Then, the flow may proceed similarly as described above with reference to updated profile 5. Specifically, the smartphone 2 may provide the patient with the feedback associated with the augmented profile 7, and at least one patient-specific value of the at least one parameter may repeatedly be retrieved from the patient 1, e.g., at least one patient-specific value may be retrieved from the patient 1, e.g., by means of a blood pressure measurement. The at least one value may be retrieved repeatedly (e.g., periodically or quasi-periodically as described above) or may be retrieved once. Based on the retrieved data, it may either be decided automatically whether the at least one feedback has led to an improvement of the patient and/or it may be determined based on an external input, e.g., by means of the medical staff and/or the patient as described above, e.g., via an electronic device in communication with the apparatus.

If the at least one metric is still above a threshold, the augmented profile 7 may either be rejected as not applicable or may be presented to the medical staff and/or the patient requesting further input. Additionally or alternatively, the patient may be presented with the advice to consult medical staff and/or the medical staff may be alerted to attend to the patient.

The sequence as described above may be executed once or may be executed repeatedly in a closed loop sequence as described above.

In an exemplary embodiment, the aspects described herein may be implemented to optimize the health state of a patient after e.g., a surgery. In such a scenario, the patient may suffer of certain health constraints which should at least partially be compensated by the present disclosure. For example, the surgery may relate to an implantation of a medical device. The apparatus may then in particular receive one or more patient-specific parameter values monitored by the medical device (e.g., pacemaker, defibrillator, etc.).

In the beginning, shortly after the surgery, a new profile may be generated for the patient. The profile may comprise characterizing information of the patient, e.g., anamnesis related parameters (as described above), a patient-ID (as described above), etc., and may be used to track the temporal evolution of patient-specific values of at least one parameter (e.g., a pulse rate).

After generating the profile for the patient, the generated profile may be compared with stored profiles (which may be assignable by means of a patient-specific ID) of the same patient (a historic profile of the patient) and/or with profiles of other patients. The comparison may involve a determination of similarities between the generated profile for the patient and the stored profiles (as described above). The stored profiles may comprise at least one feedback (e.g., a suggestion to perform a certain action such as changing eating habits and/or to change certain eating habits) associated with a patient-specific value of at least one parameter. In other words, the stored profiles may comprise a profile for a patient with a similar age, similar pre-conditions (e.g., no or the same pre-existing conditions) with feedbacks that are positively confirmed. That stored profile may be selected as described herein based on a comparison of the generated profile for the patient (preferably based on rank one similarities as described above). The initial feedback may then initially be suggested to the patient, by means of a respective smartphone app, according to the selected stored profile.

Therefore, the selection of the feedback, for suggestion a change of a habit to the patient, may be based on evidence-based profiles.

The apparatus may either forward the at least one feedback, which was gathered from the stored profile to the smartphone app by which the feedback may thus be automatically be indicated to the patient (e.g., by means of an acoustic indication (e.g., alarm) and/or e.g., push notification). The at least one feedback may additionally or alternatively be forwarded to the medical staff (e.g., via a dedicated information and/or communication system) and the at least one feedback may then be required to be confirmed by the medical staff prior to suggesting the at least one feedback to the patient.

If the at least one feedback is provided to the patient, it may be saved to the generated profile for the patient. Additionally, the generated profile for the patient may also be equipped with a positivity value (as described above). For example, if the feedback is confirmed by the patient and/or medical staff, the positivity value may be assigned with a value indicating that the feedback may be regarded as beneficial for the patient. The positivity value may further be supported by the patient-specific value of the at least one parameter.

However, it may also be possible that the medical staff and/or the patient intend a deviation from the feedback as forwarded by the apparatus. In such a case, the extracted at least one feedback may be stored in the generated profile with a positivity value which indicates that the feedback may be harmful and/or not applicable for the patient. The feedbacks suggested by the medical staff and/or the patient may be stored in the generated profile with a positivity value indicating the feedbacks as beneficial for the patient. This step supports a self-learning functionality of the present invention.

Also, if the at least one feedback is confirmed (i.e., applied by the patient) or altered during a regular check-up by medical staff and/or the patient (or after an alert), the profile may be updated and the feedbacks' positivity value may be adjusted accordingly. For example, it may be further increased, if the at least one feedback is confirmed, or it may be reduced if the at least one feedback is altered.

After the initialization of the smartphone app, the apparatus may (e.g., as described herein, for example, continuously/repeatedly, in real-time, e.g., once per second) receive patient-specific values for the at least one parameter which may be measured by a sensor system, e.g., of wearable (a smartwatch), such as a pulse rate. The received values may be used to generate updated profiles (e.g., in real-time) for the patient which may be compared to stored profiles and/or the initially generated profile. The latter may allow a tracking of the evolution of the health state of the patient and may allow an early detection of a decline of the health state of the patient, e.g., if a certain threshold (a pre-defined interval of a pulse rate) is exceeded. In the latter case, a comparison with stored profiles may possibly only follow once the updated profile (at least one parameter value) deviates from the initially generated profile (corresponding parameter value(s)) by more than a predetermined threshold. In both cases, the comparison of patient-specific values with stored respective values may allow to readjust the at least one feedback as provided to the patient by means of the smartphone app, based on stored feedbacks which have improved the health state of patients in the past.

If the repeatedly occurring receiving of the patient-specific value indicates a change of the health state of the patient which may indicate a sub-optimal health state, e.g., as determined by a comparison with the initial and/or stored profiles, the deviation may be saved to the generated profile for the patient along with a respective positivity value (indicating the sub-optimal health state). In such a case, a comparison of the generated profile for the patient with stored profiles may be performed with the goal of determining a respective feedback for the patient which is more suitable for the patient. Alternatively, upon a corresponding alert, the medical staff and/or the patient himself may change the feedback which may then be stored to the generated profile for the patient. The overwritten at least one feedback may also be stored to the generated profile for the patient, assigned with a respective positivity value which indicates that the at least one feedback has led to a non-satisfying state of the patient.

The apparatus may continue receiving patient-specific values and/or compare the received values with stored values (in the stored profiles). The received values may be stored together with the respective at least one feedback in the generated profile for the patient. If a deterioration of a patient is detected, it is also possible to revert to the initial (or any previous) feedback.

It is also conceivable that an alert is indicated to the patient and/or the medical staff, if for another profile for another patient a deterioration is detected. An alert may then be sent to the smartphone app (associated patients and/or medical staff) which were provided with similar feedbacks as stored in the profile for which a deterioration was detected.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An apparatus for optimizing a health state of a patient, the apparatus comprising:
a) means for receiving a patient-specific value of at least one parameter;
b) means for generating a profile for the patient based at least in part on the patient-specific value;
c) means for selecting a stored profile out of one or more stored profiles of the patient and/or of other patients in a storage medium, the selecting based on a comparison of the stored profile and the generated profile for the patient, wherein the stored profile comprises a stored feedback and a stored patient-specific value of the at least one parameter; and
d) means for providing feedback to patient based on the stored feedback.

2. The apparatus according to claim 1, wherein the apparatus further comprises means for selecting the stored profile based on a stored positivity value associated with the stored feedback of the stored profile.

3. The apparatus according to claim 2, wherein the means for providing the feedback is adapted such that, if the stored positivity value is negative, the feedback provided to the patient suggests to the patient to avoid an activity associated with the stored feedback.

4. The apparatus according to claim 2, wherein the means for providing the feedback is adapted such that, if the stored positivity value is positive, the feedback provided to the patient suggests an activity associated with the stored feedback.

5. The apparatus according to claim 1, further comprising:
means for obtaining a positivity value associated with the feedback provided to the patient;
means for updating the profile for the patient based on the obtained positivity value;
means for storing the updated profile for the patient in the storage medium.

6. The apparatus according to claim 5, wherein the means for obtaining the positivity value further comprises means for receiving an update of the patient-specific value of the at least one parameter and/or of a patient-specific value of at least one further parameter.

7. The apparatus according to claim 5, wherein the means for obtaining the positivity value comprises means for obtaining input from the patient.

8. The apparatus according to claim 1, wherein the means for receiving the one or more patient-specific value comprises means for repeatedly receiving updates of the at least one patient-specific value.

9. The apparatus according to claim 1, wherein the means for selecting a stored profile comprises means for calculating a metric for the stored profile, wherein the metric is calculated between the value of the at least one parameter in the generated profile for the patient and the stored patient-specific value of the at least one parameter in the stored profile.

10. The apparatus according to claim 9, wherein the means for calculating is adapted to calculate a metric for a plurality of the stored profiles, and the means for selecting is adapted to select a stored profile for which the metric is minimized.

11. The apparatus according to claim 9, wherein the means for selecting the stored profile is adapted to select the stored profile, if the metric is below a threshold.

12. The apparatus according to claim 1, wherein the apparatus is adapted to store the updated profile for the patient in a blockchain.

13. A method for optimizing a health state of a patient, the method comprising:
a) receiving a patient-specific value of at least one parameter;
b) generating a profile for the patient based at least in part on the patient-specific value;
c) selecting a stored profile out of one or more stored profiles of the patient and/or of other patients in a storage medium, the selecting based on a comparison of the stored profile and the generated profile for the patient, wherein the stored profile comprises a stored feedback and a stored patient-specific value of the at least one parameter; and
d) providing feedback to the patient based on the stored feedback.

14. The method according to claim 13, further including selecting the stored profile based on a stored positivity value associated with the stored feedback of the stored profile.

15. Computer program comprising instructions which cause a computer to implement the steps of any of claim 13, when the instructions are executed.
